(19) United States
(12) Patent Application Publication
(10) Pub. No.: US 2007/0160550 A1
(43) Pub. Date: Jul. 12, 2007

(54) Skin lightening compositions

(76) Inventors: Rebecca Louise Charles nee Newsham, Sharnbrook (GB); Paula Rachel Yates, Eaton Bay (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/587,136

(22) PCT Filed: Dec. 1, 2004
(86) PCT No.: PCT/EP04/13951

§ 371 Date: Jul. 21, 2006

(30) Foreign Application Priority Data
Jan. 24, 2004 (EP) 04250394.6

(51) Int. Cl. A61K 8/49; A61K 8/97

(52) U.S. Cl. 424/61; 424/74

(57) ABSTRACT

A skin lightening composition is provided which comprises components (a) a flavanoid, (b) vitamin C and (c) vitamin E wherein at least component (b) is provided in a form suitable for systemic administration with the other components being provided in a form suitable for topical administration.

### FIELD OF THE INVENTION

The present invention relates to agents for skin lightening, the use of these agents for inhibiting the production of melanin in the skin of a mammal and compositions suitable for such use.

### BACKGROUND TO THE INVENTION

Some people with naturally darker skin types desire to induce a degree of lightening in their overall skin colour. Skin colour is determined primarily by the amount and type of melanin, a substance which is produced within the skin by melanocytes which reside in the epidermis. Melanin is present in two forms, namely dark melanin and light melanin. Skin lightening would result if the production of dark melanin were reduced and/or the ratio of light melanin to dark melanin production were increased.

### SUMMARY OF THE INVENTION

We have now found that a combination of a flavanoid, vitamin C and vitamin E have a significant effect on inhibiting dark melanin production whilst actually increasing the production of light melanin. Skin lightening agents are usually formulated for topical use. However, we have found that if the three components are administered topically, the combination is cytotoxic. Thus we have found that at least some of the components should be administered systemically. Especially good results are obtained with split administration e.g. the flavanoid and vitamin C are administered systemically and the vitamin E is administered topically.

Accordingly, the present invention provides a skin lightening product comprising components (a) a flavanoid, (b) vitamin C and (c) vitamin E wherein at least component (b) is provided in a form suitable for systemic administration with the other components being provided in a form suitable for topical administration.

In one preferred embodiment (a) and (b) are provided in a form suitable for systemic administration and (c) is provided in a form suitable for topical administration.

In another preferred embodiment wherein components (a), (b) and (c) are provided in a form suitable for systemic administration.

Preferably the form suitable for systemic administration is an oral dosage form.

In a related aspect, the present invention provides a skin lightening product comprising a first composition for oral administration which comprises a flavanoid and vitamin C, and a second composition for topical administration which comprises vitamin E, i.e the first composition is an oral dosage form and the second composition is a topical composition.

In a further related aspect, the present invention provides a product for lightening skin which product comprises components (a) a flavanoid, (b) vitamin C and (c) vitamin E wherein at least component (b) is provided in a form suitable for systemic administration with the other components being provided in a form suitable for topical administration.

The present invention further provides a method of modulating the production of melanin in the skin of a mammal which comprises administering to said mammal (a) a flavanoid, (b) vitamin C and (c) vitamin E wherein at least component (b) is administered systemically and the other components are administered topically.

Also provided is a composition or product of the invention for use in modulating the production of melanin in the skin of a mammal as well as the use of a composition or product of the invention in the manufacture of a composition or product for inhibiting the production of melanin in the skin of a mammal.

The present invention further provides a method of increasing the ratio of light melanin to dark melanin in the skin of a mammal, the method comprising administering to said mammal, an effective amount of (a) a flavanoid, (b) vitamin C and (c) vitamin E wherein at least component (b) is administered systemically and the other components are administered topically.

In one embodiment, components (a) and (b) are administered systemically and component (c) is administered topically.

In another embodiment, components (a), (b) and (c) are administered systemically.

The present invention also provides a composition or product of the invention for use in increasing the ratio of light melanin to dark melanin in the skin of a mammal as well as the use of a composition or product of the invention for increasing the ratio of light melanin to dark melanin in the skin of a mammal. Typically such use is for cosmetic purposes.

### DETAILED DESCRIPTION OF THE INVENTION

Unless defined otherwise, all technical and scientific terms used herein have the same meaning as commonly understood by one of ordinary skill in the art.

Flavanoids

Flavanoids are polyphenolic compounds and are widely found in nature. There are several classes of flavanoids: flavanols, flavonols, flavones, isoflavones, flavanones, proanthocyanidins, anthocyanidins and hydroxystilbenes. Many of these compounds exist in glycosylated forms, especially as 0-glycosides. Typically, glycosylated forms are preferred over the aglycone. Preferably, the flavanoid is not a flavanol.

Flavonols include quercitin, kaempferol and myricetin. Flavanols include catechin, epicatechin, gallocatechin, epigallocatechin, and esters thereof with gallic acid, i.e. catechin gallate epicatechin gallate, gallocatechin gallate and epigallocatechin gallate (EGCg). Flavanones include naringenin, hesperetin and sakranetin. Flavones include luteolin and apigenin. Isoflavones include daidzein and genistein. Hydroxystilbenes include resveratrol and oxyresveratrol.

The compounds can be chemically synthesised or obtained from plant materials.

A plant extract differs from the intact plant material in that the various components present in the intact plant material will be present in different amounts in the extract, or substantially absent. Prior to extraction, plant materials may be dried and or mechanically processed, e.g. crushed.

Extracts of plant materials are typically made by solvent extraction. Solvents include “solvent” includes polar and non-polar organic solvents, water, and mixtures thereof. Preferred solvents are water, ethanol and mixtures thereof. Extraction procedures may include a heating step. Solvent extracted components may be subject to further purification/separation steps such as chromatography or fractional distillation. As used herein, “fraction” means any fractioned part of a solvent containing one or more of the active ingredients described above, e.g. obtained by chromatography or by fractional distillation.

Suitable plant sources of the various polyphenolic compounds described above include fresh fruit such as grapes (skin and seeds in particular), cranberry, blackcurrants, blackberries and citrus fruits, and vegetables such as onions, kale, broccoli and French beans.

The solubility of flavonols in aqueous solvents can be increased by co-dissolving one or more anthocyanidins (see U.S. Pat. No. 6,569,446).

In a preferred embodiment, the composition comprises a mixture of proanthocyanidins and anthocyanidins. Preferably the mixture of proanthocyanidins and anthocyanidins is provided as an extract of bark, rnore preferably an extract of the bark of French maritime pine (Pinus pinatus). One such extract is available commercially as Pycnogenol™.

In another preferred embodiment, the composition comprises one or more flavonols. Preferably, the composition comprises myricetin and/or quercetin, more preferably quercetin.

In another preferred embodiment, the composition comprises one or more flavanones. Preferably, the composition comprises naringenin, hesperitin and/or sakranetin, more preferably hesperitin.

Other compounds and/or plant extracts containing the same can be tested to confirm that they possess suitable activity using, e.g., the assay methods described in the examples.

Compositions and products of the invention may comprise mixtures of two or more flavanoids. In one embodiment, different flavanoids are provided as different plant extracts.

Vitamin C

As used herein, ‘vitamin C’ means ascorbic acid or the organic or inorganic (e.g. sodium) salts thereof. Mixtures of one or more of the acid and its salts are also included.

Vitamin E and Derivatives thereof

As used herein, “vitamin E” includes alpha-, beta-, gamma- and delta-tocopherol in any isomeric form thereof or any mixture thereof (including mixtures of isomeric forms of any of these). Derivatives of vitamin E can be oil-soluble or water-soluble. Examples of oil-soluble vitamin E derivatives, including ester derivatised vitamin E, tocopheryl acetate, tocopheryl linoleate, tocopheryl linoleate/oleate, tocopheryl nicotinate, and tocopherol (vitamin E alcohol). Water soluble vitamin E derivatives include sodium vitamin E phosphate (VEP), lauryl imino dipropionic acid tocopheryl phosphate, tocopheryl glucoside, tocopheryl succinate, tocophersolan (tocopheryl polyethylene glycol 1000 succinate), tocophereth-5, 10, 12, 18, and 50 (polyethylene glycol (PEG) tocopheryl ethers). For the PEG vitamin E derivatives, increasing numbers represent increasing numbers of PEG molecules attached to the vitamin E. Thus, as the number increases, so does water solubility, with tocopereth-5 having the lowest water solubility and tocopereth-50 having the greatest solubility in water. Derivatives of vitamin E as referred to herein have at least 50% of the biological activity of alpha-tocopherol, for example, at least 50% of the antioxidant activity of alpha-tocopherol.

Compositions and Product Forms

The skin lightening products of the present invention may be provided in forms for topical and/or systemic administration. Where the three components are provided as a split system, i.e. systemic and topical dosage forms, at least the vitamin C is provided in systemic dosage form: the topical dosage form preferably does not include any vitamin C. Preferably, in a split system, the vitamin E is provided in topical form and the flavanoid/vitamin C are provided in systemic dosage form. Alternatively, all three components are provide as a systemic dosage form.

Thus, the term “product” as used herein in the context of a product according to the present invention refers both to unitary compositions containing all essential ingredients and the situation where individual components of the overall product are split between two different compositional forms which are supplied together as a product. For example, a product may comprise one compositional form for systemic delivery of its component(s) and one compositional form for topical delivery of its component(s). Examples of products containing combinations of such compositional forms are a skin cream and a nutritional supplement tablet.

Topical Compositions

In one embodiment, the compositions of the invention comprise dosage forms formulated for topical administration, i.e. the composition/product comprises a topical composition. Accordingly, the topical compositions of the invention can be administered topically to a subject, i.e., by the direct laying on or spreading of the composition on skin. Such compositions can be prepared by combining a safe and effective amount of the active substance or substances as described above with a pharmaceutically-acceptable topical carrier or diluent, i.e. a dermatologically acceptable carrier or diluent.

The composition typically contains from about 0.1% to about 50% by weight of the active substances in total, preferably from about 1 wt % to about 50 wt %, such as from 5 or 10 wt % to about 50 wt %.

The topical compositions useful in this invention may be made into a wide variety of product types. These include, but are not limited to lotions, creams, gels, sticks, sprays, ointments and pastes. These product types may comprise several types of carrier systems including, but not limited to solutions, emulsions, gels and solids.

The topical compositions useful in this invention formulated as solutions typically include a pharmaceutically-acceptable aqueous or organic solvent. The terms “pharmaceutically-acceptable aqueous solvent” and “pharmaceutically-acceptable organic solvent” refer to a solvent which is capable of having dispersed or dissolved therein the active(s), and possesses acceptable safety properties (e.g., irritation and sensitisation characteristics). Examples of suitable organic solvents include: propylene glycol, polyethylene glycol (200-600), polypropylene glycol (425-2025), poly vinyl pyrrolidine, propylene glycol-14 butyl ether, glycerol, 1,2,4-butanetriol, sorbitol esters, 1,2,6-hexanetriol, ethanol, isopropanol, butanediol, and mixtures thereof. These solutions preferably contain from about 0.1% to about 20%, more preferably from about 1% to about 20% more preferably still from about 1% to about 10%, of each active.

If the topical compositions useful in this invention are formulated as an aerosol and applied to the skin as a spray-on, a propellant is added to a solution composition.

Topical compositions may be formulated as a solution comprising an emollient, i.e. a material used for the prevention or relief of dryness, as well as for the protection of the skin. A wide variety of suitable emollients are known and may be used herein (see Sagarin, Cosmetics, Science and Technology 2nd Edition, Vol. 1, pp. 32-43 (1972)). Such compositions preferably contain from about 2% to about 50% of a topical pharmaceutically-acceptable emollient.

If the carrier is formulated as an emulsion, preferably from about 1% to about 10%, more preferably from about 2% to about 5%, of the carrier system comprises an emulsifier. Emulsifiers may be nonionic, anionic or cationic. Suitable emulsifiers are disclosed in, for example, McCutcheon's Detergents and Emulsifiers, North American Edition, pages 317-324 (1986).

Single emulsion skin care preparations, such as lotions and creams, of the oil-in-water type and water-in-oil type are well known in the cosmetic art. Such emulsions can stabilise and enhance the penetration of actives. Multiphase emulsion compositions, such as the water-in-oil-in-water type may also be used. In general, such single or multiphase emulsions contain water, emollients and emulsifiers as essential ingredients.

Another emulsion carrier system that can be used is a micro-emulsion carrier system. Such a system comprises from about 9% to about 15% squalane; from about 25% to about 40% silicone oil; from about 8% to about 20% of a fatty alcohol; from about 15% to about 30% of polyoxyethylene sorbitan mono-fatty acid (commercially available under the trade name Tweens) or other nonionics; and from about 7% to about 20% water.

Liposomal formulations can also be used. These formulations can stabilise actives and also improve delivery of actives which do not penetrate well. Such compositions can be prepared by first combining the active with a phospholipid, such as dipalmitoylphosphatidyl choline, cholesterol and water according to the method described in Mezei & Gulasekharam, Journal of Pharmaceutics and Pharmacology, Vol. 34 (1982), pp. 473-474, or a modification thereof. Epidermal lipids of suitable composition for forming liposomes may be substituted for the phospholipid. The liposome preparation is then incorporated into one of the above topical carrier systems (for example, a gel or an oil-in-water emulsion) to produce the liposomal formulation. Other compositions and cosmetic/pharmaceutical uses of topically applied liposomes are described in Mezei, M., “Liposomes as a Skin Drug Delivery System”, Topics in Pharmaceutical Sciences (D. D. Breimer and P. Speiser, eds.), Elsevier Science Publishers B. V., New York, N.Y., 1985, pp. 345-358.

If the topical compositions are formulated as a gel or a cosmetic stick, such compositions can be formulated by the addition of a suitable amount of a thickening agent to a cream or lotion formulation.

Topical compositions may also be formulated as makeup products, such as foundations. Foundations are solution or lotion-based with appropriate amounts of thickeners, pigments and fragrance.

Various water-soluble materials may also be present in the compositions. These include humectants, proteins and polypeptides and preservatives. In addition, the topical compositions useful herein can contain conventional cosmetic adjuvants, such as dyes, opacifiers (e.g., titanium dioxide), pigments and perfumes.

The topical compositions useful in this invention may also include a safe and effective amount of a penetration enhancing agent. A preferred amount of penetration enhancing agent is from about 1% to about 5% of the composition. Examples of useful penetration enhancers are described in U.S. Pat. No. 6,068,834. Other conventional skin care product additives may also be included in the compositions. For example, collagen, hyaluronic acid, elastin, hydrolysates, primrose oil, jojoba oil, epidermal growth factor, soybean saponins, mucopolysaccharides, and mixtures thereof may be used.

It may be desirable to include in the compositions of the invention, one or more sun screening agents. A wide variety of conventional sun screening agents are disclosed in, for example, Cosmetics, Science and Technology 2nd Edition (1972), Vol. 1, Chapter VIII, pages 189 et seq. See also U.S. Pat. No. 6,068,834.

The sun screening agent must be compatible with the active(s). The composition preferably comprises from about 1% to about 20%, more preferably from about 2% to about 10%, of a sun screening agent. Exact amounts will vary depending upon the sunscreen chosen and the desired Sun Protection Factor (SPF).

An agent may also be added to any of the compositions of the invention to improve the skin substantivity of those compositions, particularly to enhance their resistance to being washed off by water, or rubbed off. A preferred agent which will provide this benefit is a copolymer of ethylene and acrylic acid. Compositions comprising this copolymer are disclosed in U.S. Pat. No. 4,663,157.

The present invention relates to methods of inhibiting melanin production in the skin of a mammal, typically a human. In one embodiment, such methods comprise the administration of a safe and effective amount of a product or composition of the invention to the skin or regions thereof. The amount of active agent and frequency of application will vary depending on the initial condition of the skin and the desired end result. Generally, the compositions should be administered in a sufficient amount and for a sufficient period of time to visibly whiten the skin.

Any dose which is less than the toxic level may be used, thus it is contemplated that for certain dosage forms, particularly topical dosage forms, the “dose” is any amount that provides the desired effect, and that amount may be so large due to frequency of application and amount applied that the maximum effective amount is irrelevant.

A safe and effective amount of active in a topical composition is applied, generally from about 1 μg to about 1 mg per cm² skin per application, preferably from about 2 μg to about 800 μg/cm² skin per application, more preferably from about 30 μg to about 700 μg/cm² skin, most preferably from about 75 μg to about 250 μg/cm² skin. Frequency of application typically ranges from about four times a day to about twice a week, more preferably from about three times a day to about once every other day, more preferably at least twice daily. It is generally preferred that at least one application occurs in the evening.

Systemic Compositions

The active agents can be combined with a pharmaceutically acceptable carrier or diluent to produce a systemic dosage form. Pharmaceutically acceptable diluents or carriers suitable for use in such compositions are well known in the art of pharmacy. The systemic dosage forms of the invention typically contain from 1 to 90% by weight of active, such as from 1 to 50% by weight of active, more preferably at least 10 or 20 wt % of active.

The systemic dosage form may consist of solid dosage forms such as tablets, hard gelatin capsules, soft gelatin capsules, bulk powders, and microcapsules of the drug. Alternately, it may consist of a liquid dosage form such as an aqueous or nonaqueous solution, emulsion, or suspension.

Solid dosage forms for oral administration are preferred compositions of the invention. Solid dosage forms are preferably prepared in unit dosage form, such as in the form of tablets and capsules. Suitably tablets may be prepared by mixing the active combination with an inert diluent such as calcium phosphate in the presence of disintegrating agents, for example maize starch, and lubricating agents, for example magnesium stearate, and tableting the mixture by known methods. Such tablets may, if desired, be provided with enteric coatings by known methods, for example by the use of cellulose acetate phthalate. Similarly, capsules, for example hard or soft gelatin capsules, containing the active combination optionally in the form of beads with or without added excipients, may be prepared by conventional means and, if desired, provided with enteric coatings in a known manner. The tablets may be formulated in a manner known to those skilled in the art so as to give a controlled release of the compound of the present invention.

Controlled release forms of the dosage forms of the present invention include rapid release formulations such as soluble granules or melt filled fast release capsules, delayed release formulations such as tablets provided with enteric coatings, for example, of cellulose acetate phthalate and, in particular, sustained release formulations. Numerous types of sustained release formulations are known to those skilled in the art. Typically, the active combination may be encapsulated within a release retarding coating, for example, a copolymer of cellulose ether and acrylate, or may be bound to small particles such as, for example, ion exchange resin beads. Alternatively, the active combination may be incorporated into a matrix containing a release retarding agent such as a hydrophilic gum e.g. xanthan gum, a cellulose derivative e.g. hydroxypropyl methylcellulose, or a polysaccharide, wax or plastics material.

The active combination may be formulated into a solid dosage form in which the two active ingredients are kept separate. For example, the dosage form may be a bilayer tablet in which the active ingredients are contained in different layers. The different layers can be formulated so as to provide the optimum release profile for each active.

Liquid fill compositions for example viscous liquid fills, liquid paste fills or thixotropic liquid fills are also suitable for oral administration. Melt filled compositions may be obtained by mixing the active combination with certain esters of natural vegetable oil fatty acids, for example, the Gelucire™ range available from Gattefosse to provide a variety of release rates. Suitably a melt-filled capsule comprises from 10 to 80% active and from 20 to 90% of a fatty acid ester excipient which comprises one or more polyol esters and triglycerides of natural vegetable oil fatty acids.

Preferably oral liquid compositions comprise from 1 to 10 wt % active together with from 1 to 50 wt % of a diluent, the remainder made up with sterile water. Optionally the composition may contain suspending agents, thickeners, cosolvents such as alcohol and/or preservatives. Suitable diluents include sweetening agents for example sorbitol, xylitol or sucrose. Suitable suspending agents or thickeners include cellulose gums, agar or natural gums, for example xanthan gum. Flavourings or other taste-masking agents known to those skilled in the art for example saccharin, sodium saccharin, acesulpham K or aspartame may be added.

Dosage forms suitable for parenteral administration can be prepared by combination of the active(s) with known pharmaceutical forms for such administration, for example sterile suspensions or sterile solutions of the active in a suitable solvent such as saline.

The preferred mode of administration is orally.

Generally, the dosage forms should be administered in a sufficient amount and for a sufficient period of time to visibly whiten the skin.

The amount of the active administered depends upon the bioavailability of the compound from the composition, in particular where oral administration is used. Typically, however, the compounds of this invention are dosed in an amount of from about 0.01 mg/kg of body weight to about 100 mg/kg, preferably from about 0.1 to about 30 mg/kg of body weight. The amount of the composition depends upon the percent of compound within its formula, which is a function of the amount of the compound required per dose, its stability, release characteristics and other pharmaceutical parameters. The doses are typically administered from once or twice weekly to one or twice daily.

The routes of administration and dosages described are intended only as a guide since a skilled practitioner will be able to determine readily the optimum route of administration and dosage for any particular individual.

Another means of systemic dosing comprises dosing any of the aforementioned compositions in a food product which does not therefore necessarily require use of a pharmacologically acceptable carrier.

As used herein, the term “food products” includes both food products as such and beverages. Suitable food products as such include spreads, dairy products (including milk and yoghurts), desserts, convenience foods/snacks, breakfast cereals and cereal bars, ready-cook meals, bread and frozen confections such as ice creams, water ices and sorbets and yoghurt ice creams. Food products also include dietary/nutritional supplements. Suitable beverages include tea, tea-flavoured drinks, coffee, soft drinks (e.g. carbonated squashes etc) and fruit juice.

The food products are typically supplemented with the active ingredients of the invention so that they contain higher amounts of the active ingredient(s) than they would normally contain.

Where split administration is used, the different dosage forms can be administered at approximately the same time or at different times. For example, the systemic dosage form may be administered with (or as) food, and the topical dosage form may be administered prior to sleep, e.g. as a night-time cream.

Uses

The products and compositions of the invention can be used to modulate melanin production in the skin of a mammal, in particular a human. More specifically, they can be used to increase the ratio of light melanin: dark melanin in skin, for example by inhibiting the production of dark melanin (eumelanin) in skin and/or increasing the production of light melanin in skin. Consequently, the compositions of the invention can be used to induce skin lightening in mammals such as humans. The advantage of increasing the ratio of light melanin: dark melanin in skin rather than simply inhibiting production of both types of melanin is that a better skin tone is produced. Typically the methods of the invention are used for cosmetic purposes.

Furthermore, age spots and areas of high pigmentation, affect skin tone and give it an uneven appearance. By boosting the light melanin fraction, it is possible to achieve a more even skin tone by increasing the ratio of light melanin to dark melanin in the age spot. This in turn will give the skin an overall lighter appearance.

Preferably the ratio of light melanin: dark melanin is increased at least two-fold relative to the control (measured as the percentage of light melanin relative to the control divided by the percentage of dark melanin relative to the control e.g. if light melanin is increased to 150% of the control and dark melanin is decreased to 50% of the control, the ratio is 3:1 relative to the control).

The present invention will now be described further with reference to the following examples which are illustrative only and non-limiting.

### EXAMPLES

In these examples, evaluation of the ability of various agents to influence levels of dark and light melanin were tested using the commercially available Melanoderm™ system.

Treatment Regime for Melanoderms™

The MelanoDerm™ MatTeks system consists of normal, human-derived epidermal keratinocytes (NHEK) and melanocytes (NHM) which have been cultured to form a multilayered, highly differentiated model of the human epidermis. The NHM within co-cultures undergo spontaneous melanogenesis leading to tissues of varying levels of pigmentation. The tissues are produced using serum free medium without artificial stimulators of melanogenesis such as TPA and IBMX. The cultures are grown on cell culture inserts at the air-liquid interface, allowing for simulated topical application of agents to be tested. Introduction of agents into the medium simulates systemic application. Thus, the model provides a useful in vitro means to evaluate agents designed to modulate skin pigmentation. It also allowed us to compare the effects of systemic versus topical administration.

On delivery, the melanoderms (MatTek MEL-300-B) were placed onto metal ring supports in a 6 well plate containing 5 ml of pre-warmed maintenance media (of EPI-100-MM-PRF), using asceptic technique as per MatTek's protocol. Incubation was carried out overnight at 37° C. and 4% CO₂ to allow the melanoderms to recover and equilibrate fully. Once placed under these conditions the MEL-300 tisue, undergo melanogenesis and differentiation.

Treatment was initiated on the following morning. Agents to be tested were dissolved in appropriate solvents and added to warmed media at final concentrations pre-assessed for melanocyte toxicity. Each time the media was changed, the spent media was aspirated from the melanoderms and reserved for testing for toxicity (Lactate Dehydrogenase (Promega) and Interleukin-1 release (R&D systems) and replaced with a fresh dose of media plus test agents whether within the media of the melanoderm. Melanoderms were returned to the incubator. This treatment regime was repeated every 48 hours until a relative difference in darkening was observed between control and test agents.

TABLE 1

| Treatment | Concentration | Supplier |
|---|---|---|
| Vitamin C | 30 μg/ml | Sigma |
| Vitamin E | 1 μM | Sigma |
| Pycnogenol | 10 μg/ml | Solgar (Pycnogenol ® |
| | | 30 mg) |
| Hesperetin | 20 μg/ml | Sigma |
| Quercetin | 10 μg/ml | Sigma |

On observation of differences in darkening, microscopic and macroscopic darkening were recorded photographically. Harvesting the tissue of the melanoderm involved cutting away the tissue from its plastic support and was followed by quantification of melanin present post-treatment versus untreated.

Selective Solubilisation of Melanin from Melanoderm Tissue

(1) Quantification of Alkali-Soluble Melanin (Light Melanin)

Melanoderm samples were cut from the plastic holders and the wet weight of tissue measured. 200 μl 1 M NaOH/8M urea was added to the melanoderm sample the tissue homogenised in a microfuge tube. Samples were whirlimixed at RT for 30 minutes on and off to release the soluble melanin. Samples were centrifuged at 13,000 rpm for 10 minutes and supernatant containing soluble melanin was removed to a fresh tube.

Protein was extracted from the supernatant by addition of 200 μl chloroform and then by mixing vigorously for 1 minute. Phases were separated by centrifugation at 13,000 rpm for 10 minutes. 150 μl of supernatant was added to a microtitre plate (in duplicate) and the OD 340 nM ascertained.

(2) Quantification of Alkali-Insoluble Melanin (Dark Melanin) 1 M NaOH was added to the remaining pellet which contains the insoluble melanin and the sample vortexed for one minute. The sample was then incubated in a water bath at 37° C. for 96 hrs with daily mixing to released the insoluble melanin. The sample was centrifuged for 10 minutes at 13,000 rpm with 200 μl of chloroform and 190 μl of the supernatant taken to a fresh tube. The supernatant was centrifuged again and 150 μl removed to a microtitre plate for analysis of absorption at 340 nm.

Calculation of Absolute Melanin Concentration

Absolute melanin is calculated as the actual melanin quantity calculated from a previously determined light melanin standard curve:
x=(y−0,003)/4.76423 (where x=concentration of melanin and y=optical density at 340 nm).

For dark melanin, the curve is x=(y−0.00553)/3.70312.

Results

TABLE 2

| | | | | Ratio of light |
|---|---|---|---|---|
| | Total Melanin | Light Melanin | Dark Melanin | melanin (μg/g):dark |
| | μg/g | μg/g | μg/g | melanin (μg/g) |
| Control | 2.55 | 1.22 | 1.34 | 0.92 |
| Pycnogenol (10 μg/ml) + Vitamin | 2.03 | 1.32 | 0.71 | 1.89 |
| C (30 μg/ml) + Vitamin | | +8.1% | −47% | |
| E (1 μM) | | | | |
| All Systemic | | | | |
| Pycnogenol (10 μg/ml) + Vitamin | Toxic to cells | | | |
| C (30 μg/ml) + Vitamin | | | | |
| E (1 μM) | | | | |
| All Topical | | | | |
| Pycnogenol (10 μg/ml) + Vitamin | 1.89 | 1.39 | 0.50 | 2.78 |
| C (30 μg/ml) + Vitamin | | +13.9% | −62.6% | |
| E (1 μM) | | | | |
| Vit E - Topical. All other | | | | |
| systemic | | | | |
| Pycnogenol (10 μg/ml) + Vitamin | Toxic to cells | | | |
| C (30 μg/ml) + Vitamin | | | | |
| E (1 μM) | | | | |
| Vit C - Topical. All other | | | | |
| systemic | | | | |
| Pycnogenol (10 μg/ml) + Vitamin | Toxic to cells | | | |
| C (30 μg/ml) + Vitamin | | | | |
| E (1 μM) | | | | |
| Vit E - systemic all other | | | | |
| topical | | | | |

We have shown that treatment of the melanoderms with Pycnogenol (10 μg/ml)+Vitamin C (30 μg/ml)+Vitamin E (1 μM) is capable of inhibiting dark melanin production and increasing the ratio of light to dark melanin and therefore capable of reducing or protecting against hyperpigmentation and/or inducing skin lightening.

However, we have found that the mode of administration is important. When Pycnogenol (10 μg/ml)+Vitamin C (30 μg/ml)+Vitamin E (1 μM) were all administered topically, the treatment appeared to be toxic to the cells. This is probably due to the ingredients being administered topically and being pro-oxidant. Significant cytotoxicity was also seen when vitamin C, or a combination of vitamin C and Pycnogenol, was administered topically and the remaining treatments administered systemically.

However, when Pycnogenol (10 μg/ml)+Vitamin C (30 μg/ml)+Vitamin E (1 μM) are all administered systemically (i.e. in the media) there is a significant decrease in dark melanin production and an increase in light melanin product (giving an increase in the ratio of light melanin to dark melanin). An alternative mode of administration is particularly effective: when Pycnogenol (10 μg/ml)+Vitamin C (30 μg/ml) are administered systemically and Vitamin E (1 μM) is administered topically, there is a significant increase in ratio of light melanin to dark melanin.

Other flavanoids were tested in place of Pycnogenol, namely hesperetin, a flavanone and quercetin, a flavonol, with similar results being obtained.

| | | | | Ratio of light |
|---|---|---|---|---|
| | Total | Light | Dark | melanin |
| | Melanin | Melanin | Melanin | (μg/g):dark |
| | μg/g | μg/g | μg/g | melanin (μg/g) |
| Control | 2.55 | 1.22 | 1.34 | 0.92 |
| Quercetin (10 μg/ml) + | 1.87 | 1.37 | 0.52 | 2.63 |
| Vitamin C (30 μg/ml) + | | +12.3% | −61.2% | |
| Vitamin E (1 μM) | | | | |
| Vit E - Topical. All other | | | | |
| systemic | | | | |
| Hesperetin (20 μg/ml) + | 2.32 | 1.40 | 0.92 | 1.52 |
| Vitamin C (30 μg/ml) + | | +14.8 | −31.3% | |
| Vitamin E (1 μM) | | | | |
| Vit E - Topical. All other | | | | |
| systemic | | | | |

The various features and embodiments of the present invention, referred to in individual sections above apply, as appropriate, to other sections, mutatis mutandis. Consequently features specified in one section may be combined with features specified in other sections, as appropriate.

All publications mentioned in the above specification are herein incorporated by reference. Various modifications and variations of the described methods and products of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in the relevant fields are intended to be within the scope of the following claims.

1. A skin lightening product comprising components (a) a flavanoid, (b) vitamin C and (c) vitamin E wherein at least component (b) is provided in a form suitable for systemic administration with the other components being provided in a form suitable for topical administration.

2. A skin lightening product according to claim 1 wherein (a) and (b) are provided in a form suitable for systemic administration and (c) is provided in a form suitable for topical administration.

3. A skin lightening product according to claim 1 wherein components (a), (b) and (c) are provided in a form suitable for systemic administration.

4. A skin lightening product according to claim 1 wherein the form suitable for systemic administration is an oral dosage form.

5. A skin lightening product according to claim 1 wherein the flavanoid is provided as a plant extract.

6. A skin lightening product according to claim 1 wherein the flavanoid is selected from quercetin, hesperetin, grape seed extract and French maritime pine bark extract.

7. A skin lightening product comprising a first composition for oral administration which comprises a flavanoid and vitamin C, and a second composition for topical administration which comprises vitamin E.

8. A method of increasing the ratio of light melanin to dark melanin in the skin of a mammal, the method comprising administering to said mammal an effective amount of (a) a flavanoid, (b) vitamin C and (c) vitamin E wherein at least component (b) is administered systemically and the other components are administered topically.

9. A method of increasing the ratio of light melanin to dark melanin in the skin of a mammal, the method comprising administering to said mammal an effective amount of (a) a flavanoid, (b) vitamin C and (c) vitamin E wherein components (a) and (b) are administered systemically and component (c) is administered topically.

10. A method of increasing the ratio of light melanin to dark melanin in the skin of a mammal, the method comprising administering systemically to said mammal an effective amount of (a) a flavanoid, (b) vitamin C and (c) vitamin E.

11. A product according to claim 1 for use in increasing the ratio of light melanin to dark melanin in the skin of a mammal.

12. Use of a product according to claim 1 for increasing the ratio of light melanin to dark melanin in the skin of a mammal.

13. A method of modulating the production of melanin in the skin of a mammal which comprises administering to said mammal (a) a flavanoid, (b) vitamin C and (c) vitamin E wherein at least component (b) is administered systemically and the other components are administered topically.

14. A product according to claim 1 for use in modulating the production of melanin in the skin of a mammal.

15. Use of a product according to claim 1 for modulating the production of melanin in the skin of a mammal.
